(19) United States
(12) Patent Application Publication
(10) Pub. No.: US 2020/0130032 A1
(43) Pub. Date: Apr. 30, 2020

(54) Automatic cleaning and recycling device for glass bottles

(71) Applicant: Shichang Lin, Fuzhou City (CN)

(72) Inventor: Shichang Lin, Fuzhou City (CN)

(21) Appl. No.: 16/726,247

(22) Filed: Dec. 24, 2019

(30) Foreign Application Priority Data
Sep. 19, 2019 (CN) 2019108861783

(51) Int. Cl. B08B 9/42; C03C 23/00; B09B 5/00; B08B 9/30; B08B 9/36; B08B 9/087; B08B 9/08; A61L 2/18

(52) U.S. Cl. CPC B08B 9/426; C03C 23/0075; B09B 5/00; B08B 9/30; B08B 9/36; A61L 2202/17; B08B 9/0826; A61L 2/18; B08B 2209/08; A61L 2202/23; B08B 9/087

(57) ABSTRACT

The invention discloses an automatic cleaning and recycling device for glass bottles, which includes a fuselage. A central cavity is opened in the fuselage. A first conveyor belt is provided near the bottom of the central cavity on the left side of the first conveyor belt. A cleaning mechanism is provided. The storage chamber is provided with a first slider, a right side of the storage chamber is provided with a fan chamber, the fan chamber is provided with a drying mechanism. The device is fixed by a cleaning mechanism and The inner wall of the glass bottle is cleaned, the outer wall of the glass bottle is cleaned by a first brush, the glass bottle is conveyed by a conveyor mechanism, the inner wall of the glass bottle is rinsed by squeezing the disinfectant and water through the first slider, and the glass bottle is quickly dried by the drying mechanism.

### CROSS-REFERENCES TO RELATED APPLICATIONS

The present application claims priority from Chinese application No. 2019108861783 filed on Sep. 19, 2019 which is hereby incorporated by reference in its entirety.

### TECHNICAL FIELD

The invention relates to the technical field of glass bottle recycling, in particular to an automatic cleaning and recycling device for glass bottles

### BACKGROUND OF THE INVENTION

Many foods are packaged in glass bottles in daily life. China is a country with a large population and consumes a large amount of food every day. Therefore, a large number of discarded glass bottles are generated. The glass bottles are directly discarded. The fragments generated after the glass bottles are broken pose a safety hazard Moreover, it will cause waste and is not conducive to the national policy of sustainable development. However, the cleaning of recycled glass bottles is tedious. The manual cleaning method requires a large amount of manpower and material resources, resulting in an increase in cost. The investment is greater than the acquisition, which violates the recycled glass The original intention of the bottle, therefore, it is necessary to design an automatic cleaning and recycling device for glass bottles.

### BRIEF SUMMARY OF THE INVENTION

The purpose of the present invention is to provide an automatic cleaning and recycling device for glass bottles, which is used to overcome the above defects in the prior art.

An automatic cleaning and recycling device for glass bottles according to an embodiment of the present invention includes a fuselage, a central cavity is opened in the fuselage, and a first conveyor belt is provided on the left side of the central cavity near the bottom. A conveyor is provided with a cleaning mechanism, which can fix and clean the inner wall of the glass bottle. The bottom wall of the middle cavity is provided with a first rotating shaft and a first brush, and the first rotating shaft and the first A brush can clean the outer wall of the glass bottle. A conveyor mechanism is provided on the right side of the middle cavity near the bottom. The conveyor mechanism can transport the glass bottles. A storage cavity is provided at the bottom of the middle cavity. A first slider, which can squeeze the disinfectant and water to rinse the inner wall of the glass bottle, a crank cavity is opened on the left side of the storage cavity, and a crank cavity is provided to control the operation of the first slider A crank mechanism, a fan cavity is provided on the right side of the storage cavity, a drying mechanism is provided in the fan cavity, the drying mechanism can dry glass bottles, and a middle cavity is provided with Mentioned cleaner Transporting conveyor means to said transport means.

Further technical description, a conveyor belt cavity is provided on the left and bottom of the middle cavity, and a first rotating shaft and two sets of transmission shafts extending up and down are rotated between the conveyor belt cavity and the middle cavity, and the bottom wall of the conveyor cavity A first motor is embedded in the first motor, and the first motor is dynamically connected to the first rotating shaft. The first rotating shaft is provided with a first pulley fixed on an outer periphery of a portion of the belt cavity. The transmission axial downward extension part on the side extends into the conveyor belt cavity and a first pulley is fixed at the end thereof. The two sets of the first pulleys are connected by a second conveyor belt, and the first motor and the A gear cavity is provided between the middle cavity, and two sets of second rotation shafts are provided between the gear cavity and the middle cavity, and the second rotation shaft and the first rotation shaft are both fixed on the outer periphery. A second gear is provided, and three sets of the second gears are meshed with each other. The first rotating shaft is fixedly provided with a first brush on an outer periphery of a portion in the middle cavity.

Further technical description, a second pulley is fixed at the end of a part of the transmission shaft in the middle cavity, and the two sets of the second pulleys are connected by a first conveyor belt, and the cleaning mechanism is fixed At the top of the first conveyor.

Further technical description, a base is fixed on the top wall of the first conveyor belt, a first chute is opened in the base, a first slide bar is slid in the first chute, and the base is provided with There are two sets of left-right symmetrical first sliding grooves. A first sliding plate is slidably arranged in the first sliding groove, and a second brush is fixed on the first sliding plate. The rod is hinged by a first link, and a second motor is fixedly embedded in the top wall of the first conveyor belt. The second motor is dynamically connected with a first screw extending upward, and the first screw and the first sliding rod A screw is connected to the top of the base, and a suction cup is rotatably mounted on the top of the first chute. A third motor is embedded in the top wall of the first chute. The third motor is dynamically connected to a first power shaft. The suction cup is fixedly connected.

Further technical description, a fourth motor is fixedly embedded in the top wall of the middle cavity, and the fourth motor is dynamically connected with a second screw, and a second slider is slidably arranged in the middle cavity. The second slider is threadedly connected, and a rotation block is provided on the bottom wall of the second slider. The rotation block is provided with a second slide cavity. The second slide cavity is provided with a second slide bar. A fifth motor is fixedly embedded in the top wall of the second sliding cavity, and a third screw is dynamically connected to the fifth motor. The third screw is screw-connected to the second sliding rod. The bottom of the rotating block is fixed. A fixing plate is provided. The fixing plate is provided with two sets of second sliding grooves, and a clamping hand is slidably arranged in the second sliding groove. The second sliding rod and the clamping hand are hinged by a second link.

Further technical description, a first small cavity is provided in the rotating block, the first small cavity penetrates the second sliding cavity, and the third screw is fixed on the outer periphery of a portion inside the first small cavity. A third gear is provided, and a first ratchet shaft extending up and down is rotated in the first small cavity, and a fourth gear is fixed on an outer periphery of a portion of the first ratchet shaft in the first small cavity. The third gear and the fourth gear are connected by a third conveyor belt. The axially extending portion of the first ratchet is controlled by a ratchet mechanism. The ratchet mechanism is dynamically connected with a second ratchet shaft. Two ratchet shafts are fixedly connected to the second slider.

Further technical description, the bottom wall on the right side of the middle cavity is provided with two sets of gear shafts extending up and down, and the axial downward extension portion of the gear on the left side projects into the belt cavity and the end is fixed A third pulley, the third pulley 78 and two sets of the first pulleys are connected by the second conveyor belt, and the gear shaft is fixedly provided with a first section on an outer periphery of a portion in the middle cavity. The four belt pulleys are connected by a fourth conveyor belt, and a bracket is evenly fixed on the outer wall of the fourth conveyor belt.

Further technical description, the storage cavity is opened at the bottom position on the right side of the middle cavity, a water inlet is opened on the rear wall of the storage cavity, and a first space is opened between the storage cavity and the middle cavity. A small cavity, a nozzle is fixed in the first small cavity, the first slider is slidably disposed in the storage cavity, a crank shaft extending up and down is rotated in the crank cavity, and the outer periphery of the crank shaft A shaft sleeve is provided for rotation, and a push rod is fixed at the right end of the sleeve. The push rod extends to the right into the storage cavity on the left and the end is hinged to the first on the left side. A slider is provided with a third link slidingly between two groups of the crank cavity, and the two sets of the first slider are fixedly connected through the third link. A transmission cavity is provided at the bottom of the crank cavity. The crank shaft extends downward into the transmission cavity, and a third rotation shaft extending up and down is rotated in the right top wall of the transmission cavity, and the third rotation shaft extends downward into the transmission cavity. A fifth pulley is fixed at the bottom end of the crank shaft and the third rotation shaft, Between the fifth via a fifth belt pulley is connected, the third rotation shaft is connected to the sixth power motor.

Further technical description, the sixth motor is fixedly embedded in the bottom wall of the fan cavity, the third rotation axially extending portion extends into the fan cavity and a fan is fixed at the end thereof, and the fan An electric heating wire is fixed on the back wall of the cavity, and a communication cavity is provided between the fan cavity and the middle cavity.

The beneficial effects of the present invention are: the device can brush the inner wall of the glass bottle by controlling the cleaning mechanism, and brush the outer wall of the glass bottle by controlling the rotation of the first brush, thereby reducing labor input and cost. Makes glass bottle recycling possible. By controlling the first slider to sterilize and rinse the glass bottle, the safety and health of the glass bottle is ensured. The glass bottle is quickly dried by the drying mechanism, which reduces the consumption time and speeds up. The efficiency of recycling is enough for a large number of recycling tasks.

### BRIEF DESCRIPTION OF THE DRAWINGS

FIG. 1 is a schematic diagram of the overall internal structure of a glass bottle automatic cleaning and recycling device according to the present invention;

FIG. 2 is a partially enlarged schematic view of A in FIG. 1 of the present invention; FIG.

3 is a partially enlarged schematic view of B in FIG. 1 of the present invention;

4 is a partially enlarged schematic diagram of C in FIG. 1 according to the present invention;

5 is a schematic diagram of a conveyor cavity in the present invention;

FIG. 6 is a schematic diagram of a stent in the present invention.

### DETAILED DESCRIPTION OF THE INVENTION

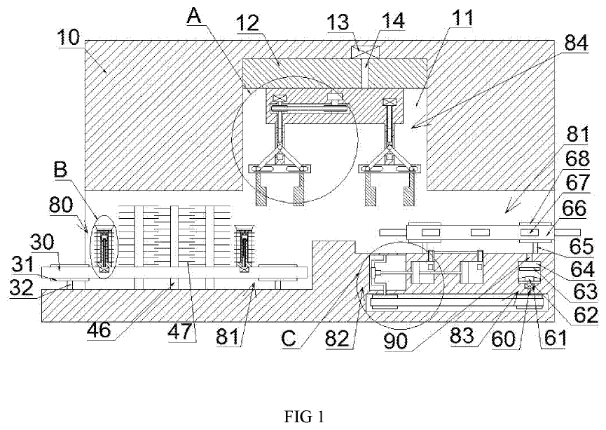

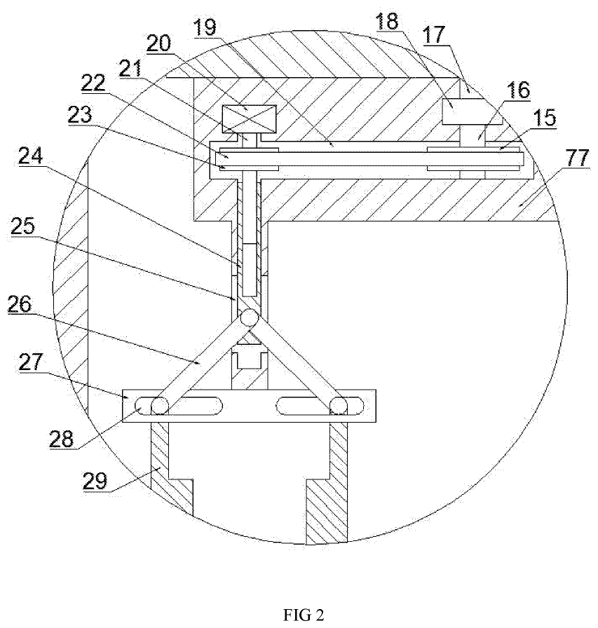

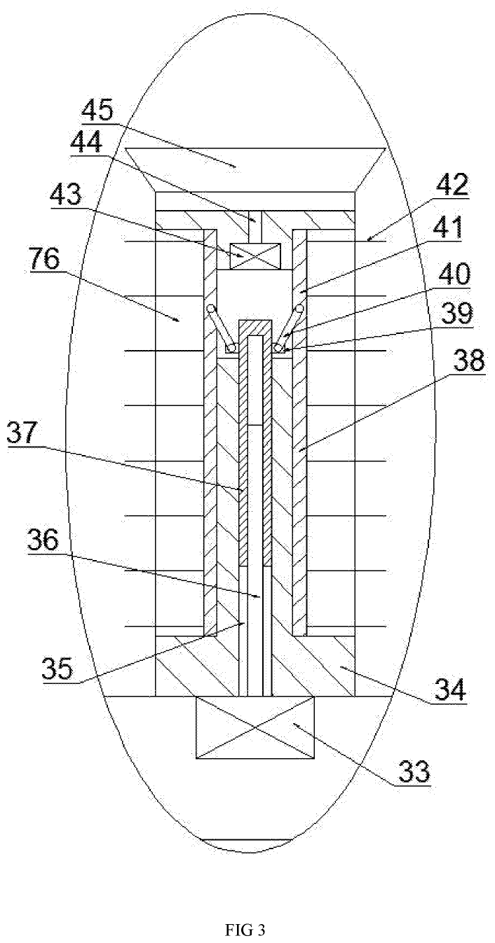

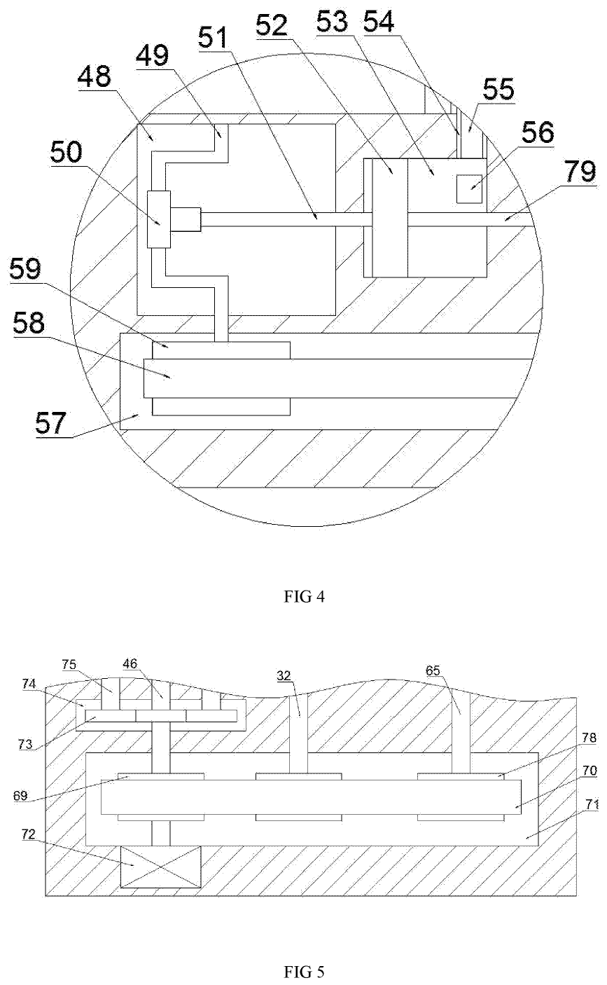

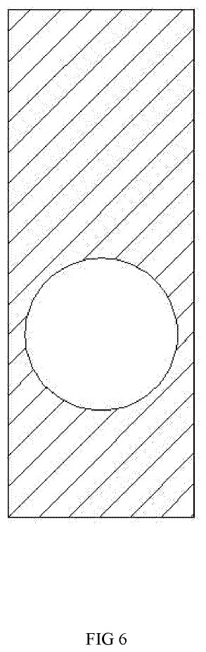

The present invention will be described in detail below with reference to FIGS. 1-6. For convenience of description, the orientation described below is defined as follows: the up-down, left-right, front-back direction described below is consistent with the up-down, left-right, front-back direction of the projection relationship of FIG. 1 itself.

Referring to FIGS. 1-6, an automatic cleaning and recycling device for glass bottles according to an embodiment of the present invention includes a fuselage 10, a central cavity 11 is opened in the fuselage 10, and a left side of the central cavity 11 is near a bottom position. A first conveyor belt 30 is provided thereon, and a cleaning mechanism 80 is provided on the first conveyor belt 30. The cleaning mechanism 80 can fix and clean the inner wall of the glass bottle, and a first rotation shaft 46 is provided on the bottom wall of the middle cavity 11. With the first brush 47, the first rotating shaft 46 and the first brush 47 can clean the outer wall of the glass bottle. A conveyor belt mechanism 81 is provided on the right side of the middle cavity 11 near the bottom, and the conveyor belt mechanism 81 A glass bottle can be transported. A storage cavity 53 is provided at the bottom of the middle cavity 11, and a first slider 52 is provided in the storage cavity 53. The first slider 52 can squeeze the disinfectant and water to rinse the inner wall of the glass bottle. A crank chamber 48 is provided on the left side of the storage chamber 53, and a crank mechanism 82 which can control the operation of the first slider 52 is provided in the crank chamber 48. A fan chamber 64 is provided on the right side of the storage chamber 53. The fan cavity 64 is provided with a drying mechanism 83. The drying mechanism 83 may Dry glass bottle, equipped with a central cavity 11 glass transport mechanism to the conveyance belt mechanism 81 from the cleaning means 80 may be 84.

Beneficially, a conveyor belt cavity 71 is provided on the left and bottom of the middle cavity 11, and a first rotation shaft 46 and two sets of transmission shafts 32 extending up and down are rotated between the conveyor belt cavity 71 and the middle cavity 11. A first motor 72 is fixedly embedded in the bottom wall of the conveyor belt cavity 71. The first motor 72 is dynamically connected to the first rotating shaft 46, and an outer periphery of a portion of the first rotating shaft 46 in the conveyor belt cavity 71. A first pulley 69 is fixed on the upper side, a downwardly extending portion of the transmission shaft 32 on the right side extends into the conveyor cavity 71 and a first pulley 69 is fixed on the end thereof. Two sets of the first pulleys are fixed. 69 is connected by a second conveyor belt 70. A gear cavity 74 is provided between the first motor 72 and the middle cavity 11, and two sets of second shafts are rotated between the gear cavity 74 and the middle cavity 11. A rotating shaft 75. A second gear 73 is fixed on the outer periphery of the second rotating shaft 75 and the first rotating shaft 46. Three groups of the second gears 73 mesh with each other. A first hair brush 47 is fixed on an outer periphery of a portion in the middle cavity 11. By controlling the operation of the first motor 72, the first The movable shaft 46 drives the second gear 73 is rotated, so that rotation of the second rotating shaft 75, the first brush 47 is rotated, the outer wall of scrubber bottles.

Advantageously, a second pulley 31 is fixed at the end of a portion of the transmission shaft 32 in the middle cavity 11, and the two sets of the second pulleys 31 are connected by a first conveyor belt 30. A cleaning mechanism 80 is fixed on the top of a conveyor belt 30. By controlling the transmission shaft 32 to rotate, the second pulley 31 drives the first conveyor belt 30 to rotate, which drives the cleaning mechanism 80 to move.

Beneficially, a base 34 is fixed on the top wall of the first conveyor belt 30. A first slide groove 35 is opened in the base 34, and a first slide bar 37 is slid in the first slide groove 35. The base 34 is provided with two sets of left-right symmetrical first sliding grooves 76. A first sliding plate 41 is slidably arranged in the first sliding groove 76, and a second brush 42 is fixedly attached to the first sliding plate 41. The first slide plate 41 and the first slide bar 37 are hinged through a first link 40. A second motor 33 is fixedly embedded in the top wall of the first conveyor belt 30. A first screw 36, the first screw 36 is screwed with the first sliding rod 37, a suction cup 45 is rotatably provided on the top of the base 34, and a third motor is fixedly embedded in the top wall of the first sliding groove 35 43. The third motor 43 is power-connected with a first power shaft 44. The first power shaft 44 is fixedly connected to the suction cup 45. By controlling the operation of the second motor 33, the first screw 36 is rotated. To raise the first slide bar 37, rotate the first link 40, slide the first slide plate 41 to the outside, and extend the second brush 42 The inner wall of the glass bottle is cleaned. The suction cup 45 can hold the glass bottle tightly. By controlling the operation of the third motor 43, the first power shaft 44 drives the suction cup 45 to rotate and rotates the glass bottle for brushing.

Beneficially, a fourth motor 13 is fixedly embedded in the top wall of the middle cavity 11, and a second screw 14 is dynamically connected to the fourth motor 13. A second slider 12 is slidably disposed in the middle cavity 11. The second screw 14 is threadedly connected to the second slide block 12. A rotation block 77 is provided on the bottom wall of the second slide block 12. A second slide cavity 25 is provided in the rotation block 77. A second sliding rod 24 is slidably disposed in the sliding cavity 25. A fifth motor 20 is fixedly embedded in the top wall of the second sliding cavity 25. The third motor 21 is connected to the fifth motor 20 by power. 21 is screwed with the second sliding rod 24, and a fixing plate 27 is fixed at the bottom of the rotating block 77, and two sets of second sliding grooves 28 are opened in the fixing plate 27. A gripper 29 is slidingly provided, and the second slide bar 24 and the gripper 29 are hinged through a second link 26. By controlling the fifth motor 20 to work, the third screw 21 is rotated to make the The second slide bar 24 is lowered, so that the two sets of the grippers 29 move toward each other inwardly along the second slide groove 28 to clamp the glass bottle.

Advantageously, a first small cavity 19 is provided in the rotating block 77, the first small cavity 19 penetrates the second sliding cavity 25, and a portion of the third screw 21 in the first small cavity 19 A third gear 23 is fixed on the outer periphery of the first small cavity 19. A first ratchet shaft 16 extending up and down is rotatably provided in the first small cavity 19. A portion of the first ratchet shaft 16 in the first small cavity 19 is rotated. A fourth gear 15 is fixed on the outer periphery, and the third gear 23 and the fourth gear 15 are connected by a third conveyor belt 22. The upwardly extending portion of the first ratchet shaft 16 is controlled and connected with a ratchet mechanism 18, so The ratchet mechanism 18 is dynamically connected with a second ratchet shaft 17, and the second ratchet shaft 17 is fixedly connected to the second slider 12. By controlling the third screw 21 to rotate forward, the third gear 23 is driven. The third conveyor belt 22 rotates, so that the fourth gear 15 drives the first ratchet shaft 16 to rotate, the ratchet mechanism 18 drives the second ratchet shaft 17 to rotate, and the rotating block 77 rotates.

Advantageously, the bottom wall on the right side of the middle cavity 11 is provided with two sets of gear shafts 65 extending up and down, and the gear shaft 65 on the left side extends downward into the conveyor cavity 71 and its ends are fixed. A third pulley 78 is provided, and the third pulley 78 and the two sets of the first pulleys 69 are connected through the second conveyor belt 70. The portion of the gear shaft 65 in the middle cavity 11 A fourth pulley 68 is fixed on the outer circumference of the fourth pulley 68. The fourth pulley 68 is connected by a fourth conveyor belt 66. A bracket 67 is evenly fixed on the outer wall of the fourth conveyor belt 66. The rotation of the pulley 69 causes the second conveyor belt 70 to rotate the third pulley 78, the gear shaft 65, the fourth pulley 68, and the fourth conveyor 66 to rotate the bracket 67. The glass bottle can be placed on the bracket 67 to complete the transportation of the glass bottle from left to right.

Beneficially, the storage cavity 53 is opened at the bottom position on the right side of the middle cavity 11. A water inlet 56 is opened on the rear wall of the storage cavity 53, and the storage cavity 53 communicates with the middle cavity 11. A first small cavity 55 is provided, and a nozzle 54 is fixed in the first small cavity 55. The first slider 52 is slidably disposed in the storage cavity 53. The crank cavity 48 is provided with a vertical extension for rotation. A crank shaft 49 is provided on the outer periphery of the crank shaft 49. A shaft sleeve 50 is rotatably provided on the right end of the shaft sleeve 50. A push rod 51 is fixed to the right end of the shaft sleeve 50. The first slider 52 on the left side is hinged in the storage cavity 53 and the end thereof. A third link 79 is slid between two groups of the crank cavity 48, and between the two groups of the first slider 52. Through the third connecting rod 79, a transmission cavity 57 is opened at the bottom of the crank cavity 48. The crank shaft 49 extends downward into the transmission cavity 57 and is located in the right top wall of the transmission cavity 57. A third rotating shaft 60 is provided to rotate up and down, the third rotating shaft 60 extends downward into the transmission cavity 57, the crank shaft 49 and the third rotation A fifth pulley 59 is fixed at the bottom end of the 60. The two sets of the fifth pulley 59 are connected by a fifth conveyor belt 58. The third rotating shaft 60 is powered by a sixth motor 61. The sixth motor 61 operates to rotate the third rotating shaft 60 to drive the fifth pulley 59 on the right side, to rotate the fifth conveyor belt 58 and to cause the fifth pulley 59 on the left side to drive the fifth pulley 59. The crank shaft 49 is rotated, so that the shaft sleeve 50 is rotated, the push rod 51 drives the first slider 52 on the left side to slide to the right, and the third link 79 drives the first side on the right side. The slider 52 slides to the right, squeezing the disinfectant and water into the inside of the glass bottle to complete the disinfection and cleaning.

Beneficially, the sixth motor 61 is fixedly embedded in the bottom wall of the fan cavity 64, the upwardly extending portion of the third rotating shaft 60 projects into the fan cavity 64, and a fan 62 is fixed at the end thereof. An electric heating wire 63 is fixed on the rear wall of the fan cavity 64, and a communication cavity 90 is opened between the fan cavity 64 and the middle cavity 11. The electric heating wire 63 is controlled to work to heat the inside of the fan cavity 64. By controlling the operation of the sixth motor 61, the third rotating shaft 60 drives the fan 62 to rotate, blows hot air to the glass bottle through the through cavity 90, and dries the glass bottle.

When in use, place the glass bottle with the opening facing down on the cleaning mechanism 80, control the suction cup 45 to hold the bottom of the inside of the glass bottle to fix it, and control the second motor 33 to work to rotate the first screw 36 and the first slide bar. 37 rises, makes the first link 40 push the first slide plate 41 to slide outward, and extends the second brush 42 to the outside. By controlling the operation of the third motor 43, the first power shaft 44 drives the suction cup 45 to rotate, so that the glass bottle Rotate to clean the inner wall by the second brush 42. By controlling the operation of the first motor 72, the first pulley 69 drives the second conveyor 70 to rotate, and the right first pulley 69 drives the drive shaft 32 to rotate, so that The second pulley 31 drives the first conveyor 30 to rotate, so that the cleaning mechanism 80 drives the glass bottle to move to the right. By controlling the first motor 72 to work, the first rotating shaft 46 drives the second gear 73 to rotate, and the second rotating shaft 75 Rotate to rotate the first brush 47 to clean the outer wall of the glass bottle that moves to the right. When the glass bottle moves directly under the third screw 21, the first motor 72 stops working, and the fourth motor 13 is controlled to work. Make the second snail 14 turns, the second slider 12 drives the rotating block 77 to lower, and the fixing plate 27 drives the gripper 29 to lower. When the gripper 29 is lowered below the bottom wall of the glass bottle, the fourth motor 13 stops working and controls the fifth motor. 20 works, rotates the third screw 21, raises the second slide rod 24, moves the clamped hands 29 of the two groups closer to each other, clamps the glass bottle, controls the fourth motor 13 to reverse, and makes the second screw 14 reverse The second slider 12 drives the second ratchet shaft 17 to rise, the fixed plate 27 drives the gripper 29 to rise, and the transport mechanism 84 is reset. By controlling the fifth motor 20 to reverse, the third screw 21 drives the third gear. 23 rotation, the third conveyor 22 drives the fourth gear 15 to rotate, the first ratchet shaft 16 is reversed, the second ratchet shaft 17 is reversed, the rotation block 77 is rotated, and the glass bottle mouth is rotated to the fixed mouth of the bracket 67 Directly above, by controlling the operation of the fourth motor 13, the second screw 14 is rotated, the second slider 12 drives the rotating block 77 to descend, and the fixing plate 27 drives the gripper 29 to descend. When the glass bottle opening is inserted into the fixing opening of the bracket 67 The fourth motor 13 stops working and controls the third screw 21 to work, The third screw 21 is rotated, the second slide bar 24 is lowered, the two sets of grippers 29 are slid to each other, the glass bottle is dropped on the bracket 67, the fourth motor 13 is controlled to be reversed, and the transport mechanism 84 is reset. The first motor 72 is operated to cause the first rotating shaft 46 to drive the first pulley 69 to rotate, the second conveyor belt 70 to drive the third pulley 78 to rotate, and the gear shaft 65 to drive the fourth pulley 68 to rotate, so that the fourth conveyor 66 Drive the bracket 67 to rotate, inject disinfectant into the storage chamber 53 on the left through the water inlet 56 and inject clean water into the storage chamber 53 on the right through the water inlet 56. By controlling the operation of the sixth motor 61, the third rotating shaft 60 Drive the fifth belt pulley 59 on the right to rotate, and the fifth belt belt 58 to rotate the fifth belt 59 on the left. The crank shaft 49 drives the sleeve 50 to rotate, and the push rod 51 drives the first slider 52 on the left. When sliding, the first slider 52 on the left drives the first slider 52 on the right to slide through the third link 79. When the first slider 52 slides to the left, the disinfectant and water flow into the storage chamber 53. When a slider 52 slides to the right, the disinfectant and water are poured into the glass bottle for disinfection and cleaning. By controlling the operation of the heating wire 63 to heat the air in the fan cavity 64, and by controlling the operation of the sixth motor 61, the third rotating shaft 60 drives the fan 62 to rotate, and the hot air is blown to the glass bottle through the cavity 90, and the glass bottle is dried.

The beneficial effects of the present invention are: the device can brush the inner wall of the glass bottle by controlling the cleaning mechanism, and brush the outer wall of the glass bottle by controlling the rotation of the first brush, thereby reducing labor input and cost. Makes glass bottle recycling possible. By controlling the first slider to sterilize and rinse the glass bottle, the safety and health of the glass bottle is ensured. The glass bottle is quickly dried by the drying mechanism, which reduces the consumption time and speeds up. The efficiency of recycling is enough for a large number of recycling tasks.

Those skilled in the art can clearly understand that various modifications to the above embodiments can be made without departing from the overall spirit and concept of the present invention. They all fall within the protection scope of the present invention. The protection scheme of the present invention is subject to the claims attached to the present invention.

1. An automatic cleaning and recycling device for glass bottles includes a fuselage, which is characterized in that: a central cavity is opened in the fuselage, and a first conveyor belt is provided at a position near the bottom of the left side of the central cavity. A cleaning mechanism is provided, which can fix and clean the inner wall of the glass bottle. The bottom wall of the middle cavity is provided with a first rotating shaft and a first brush, and the first rotating shaft and the first brush The outer wall of the glass bottle can be cleaned. A conveyor mechanism is provided on the right side of the middle cavity near the bottom. The conveyor mechanism can transport the glass bottles. A storage cavity is provided at the bottom of the middle cavity, and a first slide is provided in the storage cavity. Block, the first slider can squeeze the disinfectant and water to rinse the inner wall of the glass bottle, a crank cavity is provided on the left side of the storage cavity, and a crank mechanism is provided in the crank cavity to control the operation of the first slider A fan cavity is provided on the right side of the storage cavity, a drying mechanism is provided in the fan cavity, the drying mechanism can dry the glass bottle, and a glass bottle can be removed from the cleaning mechanism in the middle cavity. Ship to Said transport means of the conveyor means.

2. The automatic cleaning and recycling device for glass bottles according to claim 1, wherein a conveyor belt cavity is provided on the left and bottom of the middle cavity, and an up-and-down extension is provided between the conveyor cavity and the middle cavity. A first rotating shaft and two sets of transmission shafts, a first motor is embedded in the bottom wall of the conveyor belt cavity, the first motor is dynamically connected to the first rotating shaft, and the first rotating shaft is on the conveyor A first pulley is fixed on the outer periphery of the portion in the cavity, and the transmission axial downward extension portion on the right side extends into the conveyor cavity and a first pulley is fixed on the end thereof. The pulleys are connected by a second conveyor belt. A gear cavity is provided between the first motor and the middle cavity. Two sets of second rotation shafts are provided between the gear cavity and the middle cavity. A second gear is fixed on the outer periphery of the second rotary shaft and the first rotary shaft, and three sets of the second gears mesh with each other. The first rotary shaft is fixed on the outer periphery of a portion in the middle cavity. Features a first brush.

3. The automatic cleaning and recycling device for glass bottles according to claim 2, characterized in that: a second pulley is fixed on the end of a part of the transmission shaft in the middle cavity, and two sets of the second The pulleys are connected by a first conveyor belt, and the cleaning mechanism is fixed at a top position of the first conveyor belt.

4. The automatic cleaning and recycling device for glass bottles according to claim 3, wherein a base is fixed on the top wall of the first conveyor belt, and a first chute is opened in the base, and the first A first slide bar is slidably arranged in the chute, and the base is provided with two sets of left-right symmetrical first slide grooves. A first slide plate is slidably arranged in the first slide groove, and the first slide plate is fixedly provided. A second brush, the first slide plate and the first slide bar are hinged through a first link, a second motor is fixedly embedded in the top wall of the first conveyor belt, and the second motor is dynamically connected with an upwardly extending A first screw, the first screw is screw-connected to the first slide bar, a suction cup is rotatably provided on the top of the base, a third motor is fixedly embedded in the top wall of the first slide slot, and the third motor A first power shaft is dynamically connected, and the first power shaft is fixedly connected to the suction cup.

5. The automatic cleaning and recycling equipment for glass bottles according to claim 3, characterized in that: a fourth motor is fixedly embedded in the top wall of the middle cavity, and the fourth motor is powered by a second screw, and the middle A second slider is slid in the cavity, and the second screw is screw-connected to the second slider. A rotation block is provided on the bottom wall of the second slider, and a second slider is provided in the rotation block. A second sliding rod is slidably arranged in the second sliding cavity, a fifth motor is fixedly embedded in the top wall of the second sliding cavity, and the third motor is powered by a third screw, and the third screw It is screw-connected with the second slide bar, and a fixed plate is fixed at the bottom of the rotating block. Two sets of second slide grooves are opened in the fixed plate, and the second slide groove is provided with a gripper. The second slider and the gripper are hinged through a second link.

6. The automatic cleaning and recycling device for glass bottles according to claim 5, wherein a first small cavity is opened in the rotating block, the first small cavity runs through the second sliding cavity, and the first A third gear is fixed on the outer periphery of the portion of the three screws in the first small cavity, and a first ratchet shaft extending up and down is rotatably provided in the first small cavity. The first ratchet shaft is in the first cavity. A fourth gear is fixed on the outer periphery of a portion in a small cavity, and the third gear and the fourth gear are connected by a third conveyor belt. The axially extending portion of the first ratchet is controlled and connected with a ratchet mechanism. A second ratchet shaft is dynamically connected to the ratchet mechanism, and the second ratchet shaft is fixedly connected to the second slider.

7. The automatic cleaning and recycling device for glass bottles according to claim 5, characterized in that: two sets of gear shafts extending up and down are rotated on the bottom wall on the right side of the middle cavity, and the gears on the left side are axial A lower extension portion extends into the conveyor belt cavity and a third pulley is fixed at the end thereof. The third pulley 78 and the two sets of the first pulleys are connected through the second conveyor belt. The gear A fourth pulley is fixed on an outer periphery of a part of the shaft in the middle cavity, and the fourth pulleys are connected by a fourth conveyor belt, and a bracket is uniformly fixed on an outer wall of the fourth conveyor belt.

8. The automatic cleaning and recycling device for glass bottles according to claim 7, characterized in that: the storage cavity is opened at the right bottom position of the middle cavity, and a water inlet is provided on the rear wall of the storage cavity, A first small cavity is opened between the storage cavity and the middle cavity. A nozzle is fixed in the first small cavity. The first slider is slidably provided in the storage cavity. The crank cavity An inner crank is provided with a crank shaft extending up and down, a crank sleeve is rotated on an outer periphery of the crank shaft, and a push rod is fixed at the right end of the bushing, and the right extension part of the push rod extends into the left side. The left side of the first slide block is hinged in the storage cavity, and a third link is provided between the two sets of the crank cavity, and the first slide block passes between the two sets of the third slide block. A connecting rod is fixedly connected, a transmission cavity is opened at the bottom of the crank cavity, the crank shaft extends downwardly into the transmission cavity, and a third rotation shaft extending up and down is rotated in the right top wall of the transmission cavity, The third rotation axis extends downward into the transmission cavity, and the crank shaft and the A fifth pulley is fixed at the bottom end of the third rotation shaft. The two sets of fifth pulleys are connected by a fifth conveyor belt, and the third rotation shaft is powered by a sixth motor.

9. The automatic cleaning and recycling device for glass bottles according to claim 8, characterized in that: the sixth motor is fixedly embedded in the bottom wall of the fan cavity, and the third rotation axially extending portion extends into A fan is fixed in the fan cavity and its end, an electric heating wire is fixed on the rear wall of the fan cavity, and a communication cavity is opened between the fan cavity and the middle cavity.
